(19) United States
(12) Patent Application Publication
(10) Pub. No.: US 2005/0220901 A1
(43) Pub. Date: Oct. 6, 2005

(54) Methods of pharmaceutical separation from plants

(76) Inventor: Samuel Huttenbauer JR., Cincinnati, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/086,928

(22) Filed: Mar. 22, 2005

Related U.S. Application Data
(60) Provisional application No. 60/555,180, filed on Mar. 22, 2004.

(51) Int. Cl. A61K 35/78

(52) U.S. Cl. 424/725

(57) ABSTRACT

Methods for recovering plant extract from plant material comprising: chopping the plant material; exposing the plant material to a solvent system; freezing the chopped plant material in the solvent; placing the chopped plant material in the solvent into a high pressure chamber; increasing pressure in the chamber; removing mixture from the chamber; and centrifuging mixture to separate out soluble material, including the plant extract in solvent.

This application claims priority to U.S. provisional patent application No. 60/555,180, filed Mar. 22, 2004. The entire disclosure of application 60/555,180 is incorporated herein by reference.

The present invention provides compositions and methods for producing proteins in plants, particularly proteins that become biologically active. Specifically, the present invention provides for methods of harvesting and processing plants, e.g., flax or crambe plants and seeds, to extract useful proteins transgenically produced in such plants. The ultimate products typically possess therapeutic, diagnostic or industrial utility.

It is now commonplace to create genetically engineered plants, referred to as transgenic plants, which have stably inserted into their chromosomes one or more foreign gene constructions intended to express a novel or foreign protein in the transgenic plants. Techniques exist to insert genes into plant cells and to regenerate whole fertile transgenic plants from such cells. For several important commercial crop species, transgenic seeds are commercially available and are widely planted and harvested.

The most common techniques currently used for creating plant transformation vectors for plant transformations are based on manipulation and construction of the genetic material in bacterial cells followed by the transfer of the genetic materials from the bacterial cells into plant cells. As most commonly practiced, DNA incorporating a protein coding region for the protein of interest is inserted into a plant expression vector which usually includes a promoter and a transcription termination, or polyadenylation sequence, both of which work in plant cells. The combination of a promoter, protein coding sequence, and a polyadenylation sequence is referred to here as a chimeric gene construction or a plant expression cassette. The plant expression vector often also includes a selectable marker gene, or a gene that confers resistance to a chemical selection agent such as an antibiotic or herbicide. Use of such a selectable marker permits transformed plant cells to be selected from among non-transformed plant cells due to the ability of the transformed plant cells to withstand application of the chemical selection agent to the cells.

Sometimes it is desired that a transgenic plant be constructed that carries more than one foreign gene construction in its genome for more than one gene of interest. If one wants to engineer a plant to receive a series of enzymes in a cascade intended to produce an end product, one generally wants the enzymes to be produced at relatively similar levels in the cells of the plant.

Plastids of higher plants may also be used for genetic engineering. Since plastids of higher plants are maternally inherited, this offers an advantage for genetic engineering of plants for tolerance or resistance to natural or chemical conditions, such as herbicide tolerance, as these traits will not be transmitted to wild-type relatives. In addition, they can provide the high level of foreign gene expression necessary for engineered traits such as the production of pharmaceutically important proteins.

Plant plastids (chloroplasts, amyloplasts, elaioplasts, etioplasts, chromoplasts, etc.) are the major biosynthetic centers that, in addition to photosynthesis, are responsible for production of industrially important compounds such as amino acids, complex carbohydrates, fatty acids, and pigments. Plastids are derived from a common precursor known as a proplastid and thus the plastids present in a given plant species all have the same genetic content. Plant cells contain 500-10,000 copies of a small 120-160 kilobase circular genome, each molecule of which has a large (approximately 25 kb) inverted repeat. Thus, it is possible to engineer plant cells to contain up to 20,000 copies of a particular gene of interest, which potentially can result in very high levels of foreign gene expression.

### SUMMARY OF THE INVENTION

In one embodiment, the present invention provides for a transgenic plant includes an artificial genetic construction that includes, 5′ to 3′, a promoter operable in plants, a protein coding sequence, and a polyadenylation sequence. The protein coding sequence can encode a protein that includes the complete amino acid sequence of at least one protein of interest.

In one embodiment, the present invention provides for a transgenic plant that includes two or more artificial genetic constructs that each include a protein coding sequence. In an alternative embodiment, the invention provides for a transgenic plant with a construct wherein the protein coding sequence encodes the expression of two or more proteins.

The present invention also provides for a method for making transgenic plants that includes the steps of constructing a plant gene expression cassette including a promoter operable in plants and a polyadenylation sequence operable in plants, the promoter and the polyadenylation sequence operably connected to a protein coding sequence encoding a protein of interest, and transforming the plant gene cassette into a plant such that progeny of the plant produces the protein of interest in stoichiometric levels.

It is another aspect of the present invention in that a method is described for the expression of multiple proteins in stoichiometric levels in transgenic plants.

The present invention also relates to a method for the recovery of plant protein from a solution by antisolvent recrystallization using a supercritical fluid. In accordance with the present invention, a solution of protein is formed in a solvent. A supercritical antisolvent fluid is dissolved in the solution at a controlled rate to expand the solution and precipitate the protein. Examples of solvents for the protein are non-aqueous solvents include, but are not limited to, ethanol, dimethylsulfoxide, tetrahydrofuran, acetic acid, formamide, dimethylformamide, ethylene glycol, liquid polyethylene glycol, and N,N-dimethylamine. Preferred antisolvent fluids are selected from the group consisting of carbon dioxide, ethane, ethylene, sulfur hexafluoride, nitrous oxide, chlorotrifluoromethane, and monofluoromethane.

The invention also provides methods of increasing the concentration of a protein of interest within a plant or plant cell by increasing the fatty acid content with the plant or plant cell through transgenes encoding enzymes involved in the fatty acid biosynthesis pathway. Such methods may be used to stabilize and maintain the proteins produced in the plant cells so that the proteins can be harvested at a later date or to increase the concentration or proteins beyond that which would be found in the plant without such fatty acid protection.

The invention also includes vectors comprising: a) a nucleotide sequence encoding a protein of interest, b) a nucleotide sequence encoding a fatty acid of interest, operably linked to c) a promoter, as well as a host cell comprising this vector. The present invention includes a plant cell, plant or plant tissue comprising the above-described vector, wherein expression of the nucleotide sequence of the vector results in production of at least one fatty acid selected from the group consisting of a monounsaturated fatty acid and a polyunsaturated fatty acid by the plant cell, plant or plant tissue. The polyunsaturated fatty acid may be, for example, docosahexaenoic acid (DHA), M, omega-6-docosapentaenoic acid, or omega-3-docosapentaenoic acid. The invention also includes one or more proteins or fatty acids expressed by the plant cell, plant or plant tissue. Additionally, the present invention encompasses a transgenic plant comprising the above-described vector, wherein expression of the nucleotide sequence of the vector results in production of a protein of interest and a polyunsaturated fatty acid in seeds of the transgenic plant.

The invention also includes vectors comprising: a) a nucleotide sequence encoding a protein of interest, b) a nucleotide sequence encoding an enzyme of interest, operably linked to c) a promoter, as well as a host cell comprising this vector, wherein the enzyme is one or more enzymes selected form the group consisting of elongases and desaturases.

Additionally, the present invention includes a method for introducing the vector into a host cell under time and conditions sufficient for expression of elongase enzyme encoded by the nucleotide sequence; and exposing the expressed elongase enzyme to a substrate polyunsaturated fatty acid in order to convert the substrate to a product polyunsaturated fatty acid. The substrate polyunsaturated fatty acid may be selected from the group consisting of, for example, linolenic acid (GLA), stearidonic acid (STA) and arachidonic acid (M), and the product polyunsaturated fatty acid may be selected from the group consisting of, for example, DGLA, 20:4n-3, and ADA, respectively.

The method may further comprise the step of exposing the product polyunsaturated fatty acid to at least one desaturase in order to convert the product polyunsaturated fatty acid to another polyunsaturated fatty acid. The product polyunsaturated fatty acid may be selected from the group consisting of, for example, DGLA, 20:4n-3, and ADA. Another polyunsaturated fatty acid may be selected from the group consisting of, for example, M, eicosapentaenoic acid (EPA), omega-6-docosapentaenoic acid, respectively, and the at least one desaturase is A5-desaturase, with respect to production of AA or EPA, and A4-desaturase, with respect to production of omega-6-docosapentaenoic acid.

There are four major families of unsaturated fatty acids. (See Table 1.) Oleic acid is the most prevalent fatty acid in nature and can be synthesized in the body from dietary stearic acid (18:0). Palmitoleic acid can be synthesized from dietary palmitic acid (16:0). Oleic and palmitoleic acids are not essential in human nutrition because they can be formed from dietary precursors. Two fatty acids are required in the diets of humans because our bodies cannot manufacture them from dietary precursors: alpha-linolenic acid, the parent compound of the omega-3 fatty acid family, and linoleic acid, the parent compound of the omega-6 family. Arachidonic acid, a metabolite of linoleic acid, is considered an essential fatty acid (EFA) only when a linoleic acid deficiency exists. Alpha-linolenic acid and linoleic acid are converted to their respective metabolites by a series of alternating desaturations and elongations. The desaturations add a double bond by removing hydrogen, while the elongations add two carbon atoms.

TABLE 1
Nomenclature of Major Families of Fatty Acids^{a}

| Parent | Number of | Family | Structural |
|---|---|---|---|
| Compound | Double Bonds | Name^{b} | Abbreviations^{c} |
| Oleic acid | One | Omega-9 | 18:1n-9 or |
| | | (ω-9) | 18:1ω-9 |
| Palmitoleic acid | One | Omega-7 | 16:1n-7 or |
| | | (ω-7) | 16:1ω-7 |
| Linoleic acid | Two | Omega-6 | 18:2n-6 or |
| | | (ω-6) | 18:2ω-6 |
| Alpha-linoleic | Three | Omega-3 | 18:3n-3 or |
| acid^{d} | | (ω-3) | 18:3ω-3 |

^{a}Adapted from Vaisey-Genser M. in Flaxseed: Health, Nutrition and Functionality. Winnipeg. MB: Flax Council of Canada, 1994, p. 11.
^{b}The family name denotes the position of the first double bond as the number of carbon atoms from the methyl end of the fatty acid chain.
^{c}Number of carbon atoms: number of double bonds (fatty acid family).
^{d}Also designated α-linolenic acid. Alpha-linolenic acid is distinct from gamma-linolenic (γ-linolenic acid (18:3n-6)), which is an intermediate in the omega-6 metabolic pathway and is a major component of evening primrose, borage, and black currant oils.

The first step in the metabolism of both EFA families is desaturation, catalyzed by delta-6-desaturase. This step is followed by elongation, then desaturation (catalyzed by delta-5-desaturase), then elongation and, finally, desaturation (catalyzed by delta-4-desaturase). The desaturation steps tend to be slow, while the elongation steps are rapid. Thus, the tissue concentrations of gamma-linolenic acid (GLA) (18:3n-6) and stearidonic acid (18:4n-3) tend to be low, because they are formed slowly by desaturation and then quickly elongated to other metabolites.

The methods of the invention may further comprise the step of exposing the another polyunsaturated fatty acid to one or more enzymes selected from the group consisting of at least one elongase and at least one additional desaturase in order to convert the another polyunsaturated fatty acid to a final polyunsaturated fatty acid. The final polyunsaturated fatty acid may be, for example, docosahexaenoic acid (DHA), AA, omega-6-docosapentaenoic acid, or omega-3-docosapentaenoic acid. In some embodiments, the final polyunsaturated fatty acid is accumulated within the seed of the plant.

Once the gene encoding the elongase has been isolated, it may then be introduced into either a prokaryotic or eukaryotic host cell through the use of a vector, plasmid or construct. The vector, for example, a bacteriophage, cosmid or plasmid, may comprise the nucleotide sequence encoding the elongase as well as any promoter which is functional in the host cell and is able to elicit expression of the elongase encoded by the nucleotide sequence. The promoter is in operable association with or operably linked to the nucleotide sequence. (A promoter is said to be operably linked with a coding sequence if the promoter affects transcription or expression of the coding sequence.) Suitable promoters include, for example, those from genes encoding alcohol dehydrogenase, glyceraldehyde-3-phosphate dehydrogenase, phosphoglucoisomerase, phosphoglycerate kinase, acid phosphatase, T7, TP1, lactase, metallothionein, cytomegalovirus immediate early, whey acidic protein, glucoamylase, and promoters activated in the presence of galactose, for example, GALL and GAL10. Additionally, nucleotide sequences which encode other proteins, oligosaccharides, lipids, etc. may also be included within the vector as well as other regulatory sequences such as a polyadenylation signal (e.g., the poly-A signal of SV-40T-antigen, ovalalbumin or bovine growth hormone). The choice of sequences present in the construct is dependent upon the desired expression products as well as the nature of the host cell.

As noted above, once the vector has been constructed, it may then be introduced into the host cell of choice by methods known to those of ordinary skill in the art including, for example, transfection, transformation and electroporation (see Molecular Cloning: A Laboratory Manual, 2nd ed., Vol. 1-3, ed. Sambrook et al., Cold Spring Harbor Laboratory Press (1989)). The host cell is then cultured under suitable conditions permitting expression of the PUFA which is then recovered and purified.

Expression in a host cell can be accomplished in a transient or stable fashion. Transient expression can occur from introduced constructs which contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a construct that can integrate into the host genome or that autonomously replicates in the host cell. Stable expression of the gene of interest can be selected for through the use of a selectable marker located on or transfected with the expression construct, followed by selection for cells expressing the marker. When stable expression results from integration, the site of the construct's integration can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

For expression of an elongase polypeptide, functional transcriptional and translational initiation and termination regions are operably linked to the DNA encoding the elongase polypeptide. Transcriptional and translational initiation and termination regions are derived from a variety of nonexclusive sources, including the DNA to be expressed, genes known or suspected to be capable of expression in the desired system, expression vectors, chemical synthesis, or from an endogenous locus in a host cell. Expression in a plant tissue and/or plant part presents certain efficiencies, particularly where the tissue or part is one which is harvested early, such as seed, leaves, fruits, flowers, roots, etc. Expression can be targeted to that location with the plant by utilizing specific regulatory sequence such as those of U.S. Pat. Nos. 5,463,174, 4,943,674, 5,106,739, 5,175,095, 5,420,034, 5,188,958, and 5,589,379.

Alternatively, the expressed protein can be an enzyme that produces a product that may be incorporated, either directly or upon further modifications, into a fluid fraction from the host plant. Expression of an elongase gene or genes, or antisense elongase transcripts, can alter the levels of specific PUFAs, or derivatives thereof, found in plant parts and/or plant tissues. The elongase polypeptide coding region may be expressed either by itself or with other genes, in order to produce tissues and/or plant parts containing higher proportions of desired PUFAs or in which the PUFA composition more closely resembles that of human breast milk (Prieto et al., PCT publication WO 95/24494). The termination region may be derived from the 3′ region of the gene from which the initiation region was obtained or from a different gene. A large number of termination regions are known to and have been found to be satisfactory in a variety of hosts from the same and different genera and species. The termination region usually is selected as a matter of convenience rather than because of any particular property.

As noted above, a plant (e.g., glycine max (soybean) or Brassica napus (canola)), plant tissue, corn, potato, sunflower, safflower, crambe, or flax may also be utilized as a host or host cell, respectively, for expression of the elongase enzyme(s) which may, in turn, be utilized in the production of polyunsaturated fatty acids. More specifically, desired PUFAs can be expressed in seed. Methods of isolating seed oils are known in the art. Thus, in addition to providing a source for PUFAs, seed oil components may be manipulated through the expression of the elongase genes, as well as perhaps desaturase genes, in order to provide seed oils that can be added to nutritional compositions, pharmaceutical compositions, animal feeds and cosmetics. Once again, a vector which comprises a DNA sequence encoding the elongase operably linked to a promoter, will be introduced into the plant tissue or plant for a time and under conditions sufficient for expression of the elongase gene. The vector may also comprise one or more genes which encode other enzymes, for example, A4-desaturase, A5-desaturase, A6-desaturase, A8-desaturase, A9-desaturase, A10-desaturase, A112-desaturase, A13-desaturase, Δ15-desaturase, Δ17-desaturase and/or Δ19-desaturase. The plant tissue or plant may produce the relevant substrate (e.g., DGLA, GLA, STA, M, ADA, EPA, 20:4n-3, etc.) upon which the enzymes act or a vector encoding enzymes which produce such substrates may be introduced into the plant tissue, plant cell, plant, or host cell of interest. In addition, substrate may be sprayed on plant tissues expressing the appropriate enzymes. Using these various techniques, one may produce PUFAs (e.g., n-6 unsaturated fatty acids such as DGLA, M or ADA, or n-3 fatty acids such as EPA or DHA) by use of a plant cell, plant tissue, plant, or host cell of interest. It should also be noted that the invention also encompasses a transgenic plant comprising the above-described vector, wherein expression of the nucleotide sequence of the vector results in production of a polyunsaturated fatty acid in, for example, the seeds of the transgenic plant.

The substrates, as shown in FIG. 1, may be produced by the host cell either naturally or transgenically, as well as the enzymes which may be encoded by DNA sequences present in the vector, which is subsequently introduced into the host cell.

In view of the above, the present invention also encompasses a method of producing one of the elongase enzymes described above, comprising: 1) isolating the desired nucleotide sequence of the elongase cDNA; 2) constructing a vector comprising said nucleotide sequence; and 3) introducing said vector into a host cell under time and conditions sufficient for the production of the elongase enzyme.

The present invention also encompasses a method of producing polyunsaturated fatty acids comprising exposing an acid to the elongase(s) produced as above such that the elongase converts the acid to a longer polyunsaturated fatty acid. For example, when GLA is exposed to elongase, it is converted to DGLA. DGLA may then be exposed to A5-desaturase, which converts the DGLA to AA. Alternatively, elongase may be utilized to convert 18:4n-3 to 20:4n-3 which may be exposed to A5-desaturase and converted to EPA. Elongase may also be used to convert 18:3n-3 to 20:3n-3, which may be, in turn, converted to 20:4n-3 by a Δ8-desaturase. Thus, elongase may be used in the production of polyunsaturated fatty acids, which may be used, in turn, for particular beneficial purposes. (See FIG. 1 for an illustration of the many roles elongase plays in several biosynthetic pathways.)

As noted above, the enzymes encoded by the Δ5-desaturase genes and Δ6-desaturase gene of the present invention are essential in the production of highly unsaturated polyunsaturated fatty acids having a length greater than 20 and 18 carbons, respectively. As an example of the importance of the genes of the present invention, the isolated Δ5-desaturase enzymes convert DGLA to M or convert eicosatetraenoic acid to EPA. M, for example, cannot be synthesized without the Δ5-desaturase genes and enzymes encoded thereby. The isolated Δ6-desaturase enzyme of the present invention converts, for example, linoleic acid (18:2n-6) to gamma-linoleic acid (GLA) and alpha-linolenic acid to stearidonic acid (STA).

Once the gene encoding any one of the desaturase enzymes has been isolated, it may then be introduced into either a prokaryotic or eukaryotic host cell through the use of a vector or construct. The vector, for example, a bacteriophage, cosmid or plasmid, may comprise the nucleotide sequence encoding either of the Δ5-desaturase enzymes, or the Δ6-desaturase enzyme, as well as any promoter which is functional in the host cell and is able to elicit expression of the desaturase encoded by the nucleotide sequence. The promoter is in operable association with or operably linked to the nucleotide sequence. (A promoter is said to be operably linked with a coding sequence if the promoter affects transcription or expression of the coding sequence.) Suitable promoters include, for example, those from genes encoding alcohol dehydrogenase, glyceraldehyde-3-phosphate dehydrogenase, phosphoglucoisomerase, phosphoglycerate kinase, acid phosphatase, T7, TPI, lactase, metallothionein, cytomegalovirus immediate early, whey acidic protein, glucoamylase, and promoters activated in the presence of galactose, for example, GAL1 and GAL10. Additionally, nucleotide sequences that encode other proteins, oligosaccharides, lipids, etc. may also be included within the vector as well as other regulatory sequences such as a polyadenylation signal (e.g., the poly-A signal of SV40T-antigen, ovalalbumin or bovine growth hormone). The choice of sequences present in the construct is dependent upon the desired expression products as well as the nature of the host cell.

As noted above, once the vector has been constructed, it may then be introduced into the host cell of choice by methods known to those of ordinary skill in the art including, for example, transfection, transformation and electroporation (see Molecular Cloning: A Laboratory Manual, 2nd ed., Vol. 1-3, ed. Sambrook et al., Cold Spring Harbor Laboratory Press (1989)). The host cell is then cultured under suitable conditions permitting expression of the genes leading to the production of the desired PUFA, which is then recovered and purified.

Expression in a host cell can be accomplished in a transient or stable fashion. Transient expression can occur from introduced constructs which contain expression signals functional in the host cell, but which constructs do not replicate and rarely integrate in the host cell, or where the host cell is not proliferating. Transient expression also can be accomplished by inducing the activity of a regulatable promoter operably linked to the gene of interest, although such inducible systems frequently exhibit a low basal level of expression. Stable expression can be achieved by introduction of a construct that can integrate into the host genome or that autonomously replicates in the host cell. Stable expression of the gene of interest can be selected for through the use of a selectable marker located on or transfected with the expression construct, followed by selection for cells expressing the marker. When stable expression results from integration, the site of the construct's integration can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

A transgenic plant may also be used in order to express the enzyme(s) of interest (i.e., either of the two Δ5-desaturases, the Δ6-desaturase, or a combination thereof), and ultimately the PUFA(s) of interest as well as the protein(s) of interest, i.e., albumin. For expression of an elongase and desaturase polypeptides, functional transcriptional and translational initiation and termination regions are operably linked to the DNA encoding the desaturase polypeptide. Transcriptional and translational initiation and termination regions are derived from a variety of nonexclusive sources, including the DNA to be expressed, genes known or suspected to be capable of expression in the desired system, expression vectors, chemical synthesis, or from an endogenous locus in a host cell. Expression in a plant tissue and/or plant part presents certain efficiencies, particularly where the tissue or part is one which is harvested early, such as seed, leaves, fruits, flowers, roots, etc. Expression can be targeted to that location with the plant by utilizing specific regulatory sequence such as those of U.S. Pat. Nos. 5,463,174, 4,943,674, 5,106,739, 5,175,095, 5,420,034, 5,188,958, and 5,589,379.

Alternatively, the expressed protein can be an enzyme, which produces a product which may be incorporated, either directly or upon further modifications, into a fluid fraction from the host plant. Expression of a desaturase gene, or antisense desaturase transcripts, can alter the levels of specific PUFAS, or derivatives thereof, found in plant parts and/or plant tissues. The termination region may be derived from the 3′ region of the gene from which the initiation region was obtained or from a different gene. A large number of termination regions are known to and have been found to be satisfactory in a variety of hosts from the same and different genera and species. The termination region usually is selected as a matter of convenience rather than because of any particular property.

The present invention also provides methods and genetic constructs for the production of transgenic plants that can be identified visually and non-destructively. The present invention further provides transgenic plants that can be safely and specifically removed from a growing site by application of a benign chemical that is converted to a phytotoxic agent in the presence of the expressed genetic construct. The methods, genetic constructs and plants of the present invention are particularly suited for those applications related to input or output traits or the heterologous production of proteins.

The present invention provides genetic constructs comprising a conditionally lethal gene operably associated with a promoter functional in a plant cell. The gene is used to select, identify or selectively kill a plant expressing said gene.

The present invention also provides a genetic construct comprising two genes adapted for expression in a plant cell. One gene is a conditionally lethal gene. Either or both gene is operably associated with a promoter functional in a plant cell.

The genetic construct comprises a conditionally lethal gene expressed to kill the plant in response to an applied chemical formulation. Therefore, in accordance with a broad aspect of the present invention, there is provided a genetic construct comprising: a) a conditionally lethal gene adapted for expression in a plant cell and b) a novel trait gene coding for a protein, peptide or antisense RNA; the novel trait gene being adapted for expression in a plant cell and, when expressed, producing a desired phenotype.

As noted above, a plant (e.g., castor, brassica, sunflower, yellow lupine, cotton, coriander, maize, sesame, rice, crambe, flax, safflower, avocado or cucumber) or plant tissue may also be utilized as a host or host cell, respectively, for expression of the protein(s) and enzyme(s) which may, in turn, be utilized in the production of polyunsaturated fatty acids. More specifically, desired PUFAS can be expressed in seed. Methods of isolating seed oils are known in the art. A vector which comprises a DNA sequence encoding the protein of interest along with the elongase and/or desaturase operably linked to a promoter, will be introduced into the plant tissue or plant for a time and under conditions sufficient for expression of the genes. The vector may also comprise one or more genes that encode other enzymes, for example, Δ4-desaturase, elongase, Δ12-desaturase, Δ15-desaturase, Δ17-desaturase, and/or Δ19-desaturase. The plant tissue or plant may produce the relevant substrate (e.g., DGLA (in the case of Δ5-desaturase), ALA (in the case of Δ6-desaturase), etc.) upon which the enzymes act or a vector encoding enzymes which produce such substrates may be introduced into the plant tissue, plant cell or plant. In addition, substrate may be sprayed on plant tissues expressing the appropriate enzymes. Using these various techniques, one may produce PUFAs (e.g., n-6 unsaturated fatty acids such as M, or n-3 fatty acids such as EPA or STA) by use of a plant cell, plant tissue or plant. It should also be noted that the invention also encompasses a transgenic plant comprising the above-described vector, wherein expression of the nucleotide sequence of the vector results in production of a polyunsaturated fatty acid in, for example, the seeds of the transgenic plant.

The present invention also encompasses a method of producing polyunsaturated fatty acids comprising exposing an acid to the enzyme such that the desaturase converts the acid to a polyunsaturated fatty acid. For example, when 20:3n-6 is exposed to the Δ5-desaturase enzyme, it is converted to M. AA may then be exposed to elongase which elongates the AA to adrenic acid (i.e., 22:4n-6). Alternatively, Δ5-desaturase may be utilized to convert 20:4n-3 to 20:5n-3 which may be exposed to elongase and converted to (n-3)-docosapentaenoic acid. The (n-3)-docosapentaenoic acid may then be converted to DHA by use of Δ4-desaturase. Thus, Δ5-desaturase may be used in the production of polyunsaturated fatty acids, which may be used, in turn, for particular beneficial purposes.

With respect to the role of Δ6-desaturase, linoleic acid may be exposed to the enzyme such that the enzyme converts the acid to GLA. An elongase may then be used to convert the GLA to DGLA. The DGLA then may be converted to AA by exposing the DGLA to a Δ5-desaturase. As another example, ALA may be exposed to a Δ6-desaturase in order to convert the ALA to STA. The STA may then be converted to 20:4n-3 by using an elongase. Subsequently, the 20:4n-3 may be converted to EPA by exposing the 20:4n-3 to a Δ5-desaturase. Thus, the Δ6-desaturase may be used in the production of PUFAs which have may advantageous properties or may be used in the production of other PUFAs.

The invention also provides nucleic acid sequences useful in enhancing expression of a wide variety of genes, both eukaryotic and prokaryotic, in plant plastids. Furthermore, plastid expression constructs are provided which are useful for genetic engineering of plant cells and which provide for enhanced expression of the fatty acid biosynthetic enzymes in plant cell plastids. The transformed plastids should be metabolically active plastids, and are preferably maintained at a high copy number in the plant tissue of interest, most preferably the chloroplasts found in green plant tissues, such as leaves or cotyledons.

The plastid expression constructs for use in this invention generally include a plastid promoter region capable of providing for enhanced expression of a DNA sequence, a DNA sequence encoding an enzyme involved in the fatty acid biosynthetic pathway, and a transcription termination region capable of terminating transcription in a plant plastid. The plastid promoter region of the present invention is preferably linked to a ribosome binding site which provides for enhanced translation of mRNA transcripts in a plant plastid.

The plastid expression construct of this invention is preferably linked to a construct having a DNA sequence encoding a selectable marker which can be expressed in a plant plastid. Expression of the selectable marker allows the identification of plant cells comprising a plastid expressing the marker. In a preferred embodiment, vectors for transfer of the construct into a plant cell include means for inserting the expression and selection constructs into the plastid genome. This preferably comprises regions of homology to the target plastid genome that flank the constructs.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

Throughout this document, all temperatures are given in degrees Celsius, and all percentages are weight percentages unless otherwise stated. The initial definition provided for a group or term herein applies to that group or term throughout the present specification, individually or as part of another group, unless otherwise indicated.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

All publications mentioned herein are incorporated herein by reference for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

FIG. 1 shows the metabolic pathway of the omega-3 and omega-6 fatty acids.

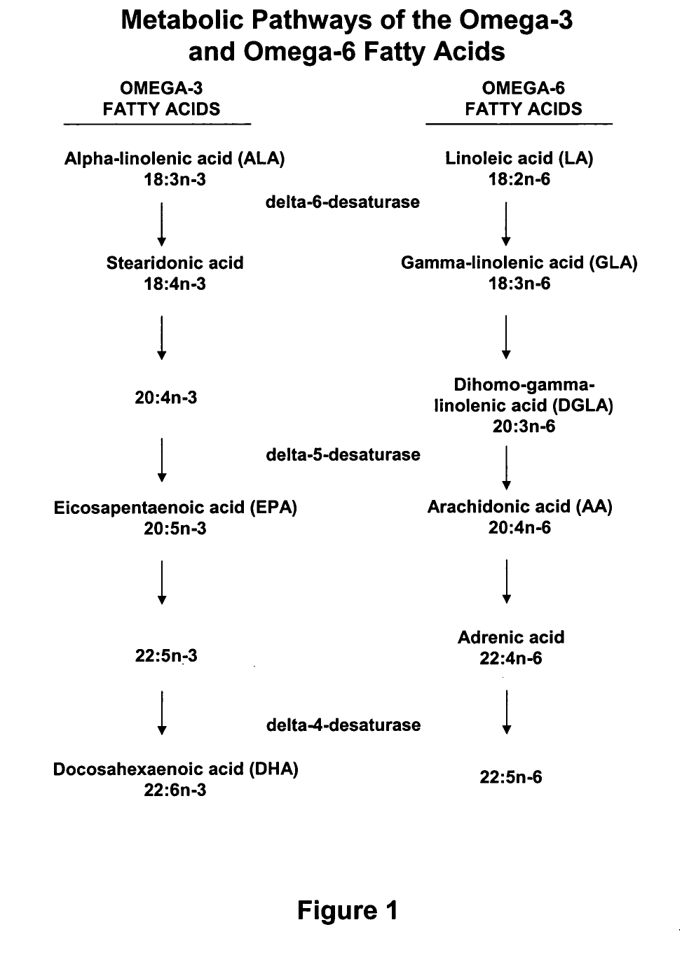

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

### DESCRIPTION OF THE EMBODIMENTS

Before the present invention is described, it is to be understood that this invention is not limited to the specific methodology, devices, formulations, and materials described as such may, of course, vary. It is also to be understood that the terminology used herein is for the purpose of describing particular embodiments only, and is not intended to limit the scope of the present invention which will be limited only by the appended claims.

The invention features methods for the production of proteins in plants.

In one embodiment, the transgenes are expressed from a seed specific promoter, and the proteins are expressed in the cytoplasm of the developing oilseed. In an alternate embodiment, enzyme and protein transgenes are expressed from a seed specific promoter and the expressed proteins are directed to the plastids using plastid targeting signals. In another embodiment, the enzyme and protein transgenes are expressed directly from the plastid chromosome where they have been integrated by homologous recombination. The enzyme and protein transgenes may also be expressed throughout the entire plant tissue from a constitutive promoter. Combinations of tissue specific and constitutive promoters with the individual genes encoding the enzymes can also be varied to alter the amount and/or location of polymer production. It is also useful to be able to control the expression of these transgenes by using promoters that can be activated following the application of an agrochemical or other active ingredient to the crop in the field. Additional control of the expression of these genes encompassed by the methods described herein include the use of recombinant technologies for targeted insertion of the transgenes into specific chromosomal sites in the plant chromosome or to regulate the expression of the transgenes.

The methods described herein involve a plant seed having a genome including (a) a promoter operably linked to a first DNA sequence and a 3′-untranslated region, wherein the first DNA sequence encodes a elongase and/or desaturase polypeptide and optionally (b) a promoter operably linked to a second DNA sequence and a 3′-untranslated region, wherein the second DNA sequence encodes a protein or interest.

DNA constructs useful in the methods described herein include transformation vectors capable of introducing transgenes into plants. Several plant transformation vector options are available, including those described in “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga, et al. eds.) Cold Spring Laboratory Press, New York (1995), which are incorporated herein by reference. Plant transformation vectors generally include one or more coding sequences of interest under the transcriptional control of 5′ and 3′ regulatory sequences, including a promoter, a transcription termination and/or polyadenylation signal, and a selectable or screenable marker gene. The usual requirements for 5′ regulatory sequences include a promoter, a transcription termination and/or a polyadenylation signal. For the expression of two or more polypeptides from a single transcript, additional RNA processing signals and ribozyme sequences can be engineered into the construct (U.S. Pat. No. 5,519,164). This approach has the advantage of locating multiple transgenes in a single locus, which is advantageous in subsequent plant breeding efforts. An additional approach is to use a vector to specifically transform the plant plastid chromosome by homologous recombination (U.S. Pat. No. 5,545,818), in which case it is possible to take advantage of the prokaryotic nature of the plastid genome and insert a number of transgenes as an operon.

A large number of plant promoters are known and result in either constitutive, or environmentally or developmentally regulated expression of the gene of interest. Plant promoters can be selected to control the expression of the transgene in different plant tissues or organelles for all of which methods are known to those skilled in the art (Gasser & Fraley, Science 244:1293-99 (1989)). The 5′ end of the transgene may be engineered to include sequences encoding plastid or other subcellular organelle targeting peptides linked in-frame with the transgene. Suitable constitutive plant promoters include the cauliflower mosaic virus ³⁵S promoter (CaMV) and enhanced CaMV promoters (Odell et. al., Nature, 313: 810 (1985)), actin promoter (McElroy et al., Plant Cell 2:163-71 (1990)), Adhl promoter (Fromm et. al., Bio/Technology 8:833-39 (1990); Kyozuka et al., Mol. Gen. Genet. 228:40-48 (1991)), ubiquitin promoters, the Figwort mosaic virus promoter, mannopine synthase promoter, nopaline synthase promoter and octopine synthase promoter. Useful regulatable promoter systems include spinach nitrate-inducible promoter, heat shock promoters, small subunit of ribulose biphosphate carboxylase promoters and chemically inducible promoters (U.S. Pat. No. 5,364,780 to Hershey et al.).

In a preferred embodiment of the methods described herein, the transgenes are expressed only in the developing seeds. Promoters suitable for this purpose include the napin gene promoter (U.S. Pat. Nos. 5,420,034 and 5,608,152), the acetyl-CoA carboxylase promoter (U.S. Pat. Nos. 5,420,034 and 5,608,152), 2S albumin promoter, seed storage protein promoter, phaseolin promoter (Slightom et. al., Proc. Natl. Acad. Sci. USA 80:1897-1901 (1983)), oleosin promoter (Plant et. al., Plant Mol. Biol. 25:193-205 (1994); Rowley et al., Biochim. Biophys. Acta. 1345:1-4 (1997); U.S. Pat. No. 5,650,554; and PCT WO 93/20216), zein promoter, glutelin promoter, starch synthase promoter, and starch branching enzyme promoter.

The transformation of suitable agronomic plant hosts using these vectors can be accomplished with a variety of methods and plant tissues. Representative plants useful in the methods disclosed herein include the Brassica family including napus, rappa, sp. carinata and juncea; maize; soybean; cottonseed; sunflower; palm; coconut; safflower; peanut; mustards including Sinapis alba; crambe; and flax. Crops harvested as biomass, such as silage corn, alfalfa, or tobacco, also are useful with the methods disclosed herein. Representative tissues for transformation using these vectors include protoplasts, cells, callus tissue, leaf discs, pollen, and meristems. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga, et al. eds.) Cold Spring Laboratory Press, New York (1995)).

In order to generate transgenic plants using the constructs described herein, the following procedures can be used to obtain a transformed plant expressing the transgenes subsequent to transformation: select the plant cells that have been transformed on a selective medium; regenerate the plant cells that have been transformed to produce differentiated plants; select transformed plants expressing the transgene at such that the level of desired polypeptide is obtained in the desired tissue and cellular location.

For the specific crops useful for practicing the described methods, transformation procedures have been established, as described for example, in “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga, et al. eds.) Cold Spring Laboratory Press, New York (1995).

Brassica napus can be transformed as described, for example, in U.S. Pat. Nos. 5,188,958 and 5,463,174. Other Brassica such as rappa, carinata and juncea as well as Sinapis alba can be transformed as described by Moloney et. al., Plant Cell Reports 8:23842 (1989). Soybean can be transformed by a number of reported procedures (U.S. Pat. Nos. 5,015,580; 5,015,944; 5,024,944; 5,322,783; 5,416,011; and 5,169,770). Several transformation procedures have been reported for the production of transgenic maize plants including pollen transformation (U.S. Pat. No. 5,629,183), silicon fiber-mediated transformation (U.S. Pat. No. 5,464,765), electroporation of protoplasts (U.S. Pat. Nos. 5,231,019; 5,472,869; and 5,384,253) gene gun, (U.S. Pat. Nos. 5,538,877 and 5,538,880 and Agrobacterium-mediated transformation (EP 0 604 662 A1; PCT WO 94/00977). The Agrobacterium-mediated procedure is particularly preferred, since single integration events of the transgene constructs are more readily obtained using this procedure, which greatly facilitates subsequent plant breeding. Cotton can be transformed by particle bombardment (U.S. Pat. Nos. 5,004,863 and 5,159,135). Sunflower can be transformed using a combination of particle bombardment and Agrobacterium infection (EP 0 486 233 A2; U.S. Pat. No. 5,030,572). Flax can be transformed by either particle bombardment or Agrobacterium-mediated transformation. Recombinase technologies include the cre-lox, FLP/FRT, and Gin systems. Methods for utilizing these technologies are described for example in U.S. Pat. No. 5,527,695 to Hodges et al.; Dale & Ow, Proc. Natl. Acad. Sci. USA 8: 10558-62 (1991); Medberry et. al., Nucleic Acids Res. 23:485-90 (1995).

Selectable marker genes useful in practicing the methods described herein include the neomycin phosphotransferase gene nptII (U.S. Pat. Nos. 5,034,322 and 5,530,196), hygromycin resistance gene (U.S. Pat. No. 5,668,298), bar gene encoding resistance to phosphinothricin (U.S. Pat. No. 5,276,268). EP 0 530 129 A1 describes a positive selection system which enables the transformed plants to outgrow the non-transformed lines by expressing a transgene encoding an enzyme that activates an inactive compound added to the growth media. Screenable marker genes useful in the methods herein include the .beta.-glucuronidase gene (Jefferson et. al., EMBO J. 6:3901-07 (1987); U.S. Pat. No. 5,268,463) and native or modified green fluorescent protein gene (Cubitt et. al., Trends Biochem Sci. 20:448-55 (1995); Pang et. al., Plant Physiol. 112:893-900 (1996)). Some of these markers have the added advantage of introducing a trait, such as herbicide resistance, into the plant of interest, thereby providing an additional agronomic value on the input side.

In one embodiment of the methods described herein, more than one gene product is expressed in the plant. This expression can be achieved via a number of different methods, including (1) introducing the encoding DNAs in a single transformation event where all necessary DNAs are on a single vector; (2) introducing the encoding DNAs in a co-transformation event where all necessary DNAs are on separate vectors but introduced into plant cells simultaneously; (3) introducing the encoding DNAs by independent transformation events successively into the plant cells i.e. transformation of transgenic plant cells expressing one or more of the encoding DNAs with additional DNA constructs; and (4) transformation of each of the required DNA constructs by separate transformation events, obtaining transgenic plants expressing the individual proteins and using traditional plant breeding methods to incorporate the entire pathway into a single plant.

The present invention provides for the expression of human serum albumin in a plant, e.g., Brassica family including napus, rappa, sp. carinata and juncea; maize; soybean; cottonseed; sunflower; palm; coconut; safflower; peanut; mustards including Sinapis alba; crambe; and flax. Stabilization of this expression can be effected by the simultaneous expression of additional genes in the plant, resulting in the production of docosahexaenoic acid (DHA).

These genes are (a) one or more polypeptides having fatty acid desaturase activity, and (b) one or more polypeptides having elongase activity. Preferably, the polypeptide having fatty acid desaturase activity has n-3 fatty acid desaturase activity and/or delta-5 fatty acid desaturase activity.

Docosahexaenoic acid, because of its extremely high affinity for albumin, appears to effectively sterically inhibit intracellular protease-mediated degradation. Accordingly, one aspect of the present invention is directed to a plant containing a recombinant nucleic acid molecule, or expression unit, containing from 5′ to 3′, a transcription initiator and a at least one structural gene. In some embodiments, the transcription initiator is a promoter functional in a plant cell (although is not necessarily naturally found in a plant). The transcription initiator may additionally comprise enhancer sequences or other regulatory elements for modulating the degree of expression and/or specificity of expression (e.g., providing temporal and/or spatial regulation of transcription). In one embodiment, the structural genes encode subunits of a multi-subunit protein.

In other embodiments, the constructs and methods of the present invention may be modified in such a way that the structural gene encoding human serum albumin (HSA) is introduced into the plant cell separately, e.g., after the introduction of the construct containing the structural genes encoding processing protein(s). Thus, a “host” processing plant is prepared and may be propagated until the expression unit comprising the albumin gene in introduced.

When proteins are synthesized in a cell they can be targeted to specific sub-cellular or extracellular locations by virtue of targeting sequences. In some cases the sequence of amino acids is synthesized as the amino terminal portion of the polypeptide and is cleaved by proteases after or during the translocation or localization process. For instance, the model of the protein secretion pathway in eukaryotes is that following ribosome binding to mRNA and initiation of translation the nascent polypeptide chain emerges. If it is a protein destined for secretion, the emerging amino terminus of the protein is recognized by signal recognition particle (SRP) that bring about a temporary stalling of translation while the mRNA, ribosome and SRP complex docks with the endoplasmic reticulum (ER). After docking, translation resumes, although now the polypeptide chain is co-translationally translocated through to the ER lumen. It is possible that proteins are translocated post-translationally; however, this process in vivo is far less efficient and generally is not considered the normal route of entry into the ER. U.S. Pat. No. 5,474,925 describes an expression construct utilizing a signal peptide translationally fused to a recombinant protein that targets the protein to the cellulose matrix of the cell wall. Thus, in one embodiment of the invention, the expression unit may comprise a structural gene fused in frame to a sequence encoding such a signal peptide.

In another aspect, proteins may be targeted to the interstitial fluids of a plant permitting a protein, such as an antibody, preferably, a monoclonal antibody, to be isolated directly from the interstitial fluids. One exemplary way of isolating proteins from interstitial fluids is described in U.S. Pat. No. 6,284,875. Thus, in one embodiment the expression unit may comprise a structural gene fused in frame to a targeting sequence from a protein secreted into interstitial fluids.

In some embodiments, the structural genes include targeting peptides for directing the expression product to a secretory pathway. To accomplish this in plants, the genes are synthesized (e.g., cloned) having either their native mammalian signal peptide encoding region, or as a fusion in which a plant secretion signal peptide is substituted. The fusion between the signal peptide and the protein should be such that upon processing by the plant, the resultant amino terminus of the protein is identical to that which is generated in the human host.

Targeting proteins to the endomembrane system of a plant is a preferred embodiment of the present invention as it provides for the proper maturation of the amino terminus of the protein. Further localization to specific regions of the endomembrane system can be accomplished if the protein of interest either has or is engineered to contain additional targeting information.

Targeting to organelles such as plastids (e.g., chloroplast) and mitochondria is also advantageous for achieving the desired amino-terminal maturation as targeting to either of these locations is dictated by an amino-terminal signal sequence that subsequently undergoes a cleavage event. In preferred embodiments, the signaling peptides direct the expression products to a plastid (e.g., a chloroplast) or other subcellular organelle. An example is the transit peptide of the small subunit of the alfalfa ribulose-biphosphate carboxylase.

In photosynthetic leaf cells of higher plants, the most conspicuous organelles are the chloroplasts, which exist in a semi-autonomous fashion within the cell, containing their own genetic system and protein synthesis machinery, but relying upon a close cooperation with the nucleo-cytoplasmic system in their development and biosynthetic activities. The chloroplast present in leaf cells is one developmental stage of this organelle. Proplastids, etioplasts, amyloplasts, and chromoplasts are other stages of this organelle. The embodiments of this invention apply to the organelle that includes chloroplast and its developmental stages.

Methods for transferring and integrating a DNA molecule into the plant host genome are well known. Methods such as Arabidopsis vacuum-infiltration or dipping are preferred because many plants can be transformed in a small space, yielding a large amount of seed to screen for transformants. Agrobacterium typically transfers a linear DNA fragment (T-DNA) with defined ends (T-DNA borders) making it a preferred method as well. Direct DNA transformation, such as microinjection, chemical treatment, or microprojectile bombardment (biolistics) is also useful. Barring any limitations on the size of the recombinant DNA construct, polycistronic gene encoding sequences according to the invention can be delivered into plants using viral vectors. The plant cells transformed may be in the form of protoplasts, cell culture, callus tissue, suspension culture, leaf, pollen or meristem.

The transformed cells may then in suitable cases be regenerated into whole plants in which the new nuclear material is stably incorporated into the genome. Both transformed monocotyledonous and dicotyledonous plant may be obtained in this way. There are a variety of plant types that can be transformed with the nucleic acid constructs of the present invention. Examples of other genetically modified plants which may be produced include field crops, cereals, fruit and vegetables such as canola, tobacco, sugarbeet, cotton, soy, maize, wheat, barley, rice, sorghum, tomatoes, mangoes, peaches, apples, pears, strawberries, bananas, melons, potatoes, carrot, lettuce, cabbage, onion, flax, and crambe. Preferred plants are Arabidopsis, Brassica species, maize, alfalfa, soybean, tobacco, crucifera, cottonseed, sunflower, legumes, flax, and crambe.

Any of the transgenic plants of the present invention may be cultivated to isolate the desired protein they contain. After cultivation, the transgenic plant is harvested to recover or express the proteins. This harvesting step may consist of harvesting the entire plant, or only the leaves, or roots of the plant. This step may either kill the plant or if only the portion of the transgenic plant is harvested may allow the remainder of the plant to continue to grow. The transgenic plants according to this invention can also be used to develop hybrids or novel varieties embodying the desired traits. Such plants would be developed using traditional selection type breeding. The transgenic plants according to this invention can be used to develop hybrids or novel varieties embodying the desired traits. Such plants would be developed using traditional selection type breeding.

After harvesting, protein isolation may be performed using methods routine in the art. For example, at least a portion of the plant may be homogenized, and the protein extracted and further purified. Extraction may comprise soaking or immersing the homogenate in a suitable solvent. As discussed above, proteins may also be isolated from interstitial fluids of plants, for example, by vacuum infiltration methods, as described in U.S. Pat. No. 6,284,875, incorporated herein by reference.

Purification methods include, but are not limited to, immuno-affinity purification and purification procedures based on the specific size of a protein/protein complex, electrophoretic mobility, biological activity, and/or net charge of the protein to be isolated.

Another aspect of the invention is directed to nucleic acid constructs comprising a nucleotide sequence that encodes at least one functionally active desaturase, wherein the nucleotide sequence is operably associated with a promoter functional in a plant cell.

Another aspect of the invention is directed to expression cassettes (also termed herein “chimeric genes”) and expression vectors comprising a promoter which directs seed-specific expression operably linked to the coding sequence of a heterologous gene such that the regulatory element is capable of controlling expression of the product encoded by the heterologous gene.

As used herein, the term “cassette” refers to a nucleotide sequence capable of expressing a particular gene if said gene is inserted so as to be operably linked to one or more regulatory regions present in the nucleotide sequence. Thus, for example, the expression cassette may comprise a heterologous coding sequence that is desired to be expressed in a plant or seed. The expression cassettes and expression vectors of the present invention are therefore useful for directing seed-specific expression of any number of heterologous genes.

Accordingly, the present invention provides chimeric genes comprising sequences of a promoter, which directs expression including the promoter, which are operably linked to a sequence encoding a heterologous gene such as a lipid metabolism enzyme. Examples of lipid metabolism genes useful for practicing the present invention include lipid desaturases such as Δ5-desaturases, Δ6-desaturases, Δ12-desaturases, Δ15-desaturases and other related desaturases such as stearoyl-ACP desaturases, acyl carrier proteins (ACPs), thioesterases, acetyl transacylases, acetyl-coA carboxylases, ketoacyl-synthases, malonyl transacylases, and elongases. Such lipid metabolism genes have been isolated and characterized from a number of different bacteria and plant species. Their nucleotide coding sequences as well as methods of isolating such coding sequences are disclosed in the published literature and are widely available to those of skill in the art.

The present invention also encompasses a method of producing polyunsaturated fatty acids within a transgenic plant comprising exposing an acid to the Δ5-desaturase enzyme such that the desaturase converts the acid to a polyunsaturated fatty acid. For example, when 20:3n-6 is exposed to Δ5-desaturase enzyme, it is converted to AA. AA may then be exposed to elongase, which elongates the AA to adrenic acid (i.e., 22:4n-6). Alternatively, A5-desaturase may be utilized to convert 20:4n-3 to 20:5n-3 which may be exposed to elongase and converted to (n-3)-docosapentaenoic acid. The (n-3)-docosapentaenoic acid may then be converted to DHA by use of Δ4-desaturase. Thus, Δ5-desaturase may be used in the production of polyunsaturated fatty acids that may be used, in turn, for particular beneficial purposes within a transgenic plant.

For production of PUFAs, depending upon the host cell, the availability of substrate, and the desired end product(s), several polypeptides, particularly desaturases, are of interest including those polypeptides which catalyze the conversion of stearic acid to oleic acid, LA to GLA, of ALA to SDA, of oleic acid to LA, or of LA to ALA, oleic acid to taxolic acid, LA to pinolenic acid, oleic acid to 6,9-actadeca-dienoic acid which includes enzymes which desaturate at the Δ6, Δ9, Δ5, Δ12, Δ15, Δ5, or Ω3 positions. Considerations for choosing a specific polypeptide having desaturase activity include the pH optimum of the polypeptide, whether the polypeptide is a rate limiting enzyme or a component thereof, whether the desaturase used is essential for synthesis of a desired poly-unsaturated fatty acid, and/or co-factors required by the polypeptide.

Two main families of poly-unsaturated fatty acids (PUFAs) are the Ω3 fatty acids, exemplified by arachidonic acid, and the Ω6 fatty acids, exemplified by eicosapentaenoic acid. PUFAs are important components of the plasma membrane of the cell, where they may be found in such forms as phospholipids and triglycerides. PUFAs also serve as precursors to other molecules of importance in human beings and animals, including the prostacyclins, leukotrienes and prostaglandins. PUFAs are necessary for proper development, particularly in the developing infant brain, and for tissue formation and repair.

Four major long chain PUFAs of importance include docosahexaenoic acid (DHA) and eicosapentaenoic acid (EPA), which are primarily found in different types of fish oil, gamma-linolenic acid (GLA), which is found in the seeds of a number of plants, including evening primrose (Oenothera biennis), borage (Borago officinalis) and black currants (Ribes nigrum), and stearidonic acid (SDA), which is found in marine oils and plant seeds.

A number of enzymes are involved in PUFA biosynthesis. Linoleic acid (LA, 18:2 Δ9,12) is produced from oleic acid (18:1 Δ9) by a Δ12-desaturase. GLA (18:3 Δ6,9,12) is produced from linoleic acid (LA, 18:2 Δ9,12) by a Δ6-desaturase. ARA (20:4 Δ5,8,11,14) production from DGLA (20:3 Δ8,11,14) is catalyzed by a Δ5-desaturase. However, animals cannot desaturate beyond the Δ9 position and therefore cannot convert oleic acid (18:1 Δ9) into linoleic acid (18:2 Δ9,12). Likewise, α-linolenic acid (ALA, 18:3 Δ9,12,15) cannot be synthesized by mammals. Other eukaryotes, including fungi and plants, have enzymes that desaturate at positions Δ12 and Δ15. The major poly-unsaturated fatty acids of animals therefore are either derived from diet and/or from desaturation and elongation of linoleic acid (18:2 Δ9,12) or linolenic acid (18:3 Δ9,12,15).

Production of gamma-linolenic acid by a Δ6-desaturase is described in U.S. Pat. No. 5,552,306 and U.S. Pat. No. 5,614,393. Production of 8,11-eicosadienoic acid using Mortierella alpina is disclosed in U.S. Pat. No. 5,376,541. Production of docosahexaenoic acid by dinoflagellates is described in U.S. Pat. No. 5,407,957. Cloning of a Δ6-desaturase from borage is described in PCT publication WO 96/21022. Cloning of Δ9-desaturases is described in the published patent applications PCT WO 91/13972, EP 0 550 162 A1, EP 0 561 569 A2, EP 0 644 263 A2, and EP 0 736 598 A1, and in U.S. Pat. No. 5,057,419. Cloning of Δ12-desaturases from various organisms is described in PCT publication WO 94/11516 and U.S. Pat. No. 5,443,974. Cloning of Δ15-desaturases from various organisms is described in PCT publication WO 93/11245. A Δ6 palmitoyl-acyl carrier protein desaturase from Thumbergia alata and its expression in E. coli is described in U.S. Pat. No. 5,614,400. Expression of a soybean stearyl-ACP desaturase in transgenic soybean embryos using a 35S promoter is disclosed in U.S. Pat. No. 5,443,974.

In particular, the desaturase and elongase genes disclosed in U.S. Pat. Nos. 6,432,684, 6,075,183, and 6,403,349 and U.S. Pat. Appl. No. 20030104596 and incorporated herein by reference, are contemplated as lipid metabolism genes particularly useful in the practice of the present invention.

To provide regulated expression of the heterologous or native genes, plants are transformed with the chimeric gene constructions of the invention. Methods of gene transfer are well known in the art. The chimeric genes can be introduced into plants by leaf disk transformation-regeneration procedure as described by Horsch et al. (1985) Science, 227; 1229-1231. Other methods of transformation such as protoplast culture (Horsch et al. (1984) Science, 223; 496; DeBlock et al. (1984) EMBO J., 2; 2143; Barton et al. (1983) Cell, 32; 1033) can also be used and are within the scope of this invention. In a preferred embodiment, plants are transformed with Agrobacterium-derived vectors such as those described in Kieft et al. (1987) Annu. Rev. Plant Physiol., 38; 467. Other well-known methods are available to insert the chimeric genes of the present invention into plant cells. Such alternative methods include biolistic approaches (Klein et al. (1987) Nature, 327; 70), electroporation, chemically-induced DNA uptake, and use of viruses or pollen as vectors.

Expression can be targeted to that location within the plant by using specific regulatory sequences, such as those of U.S. Pat. No. 5,463,174, U.S. Pat. No. 4,943,674, U.S. Pat. No. 5,106,739, U.S. Pat. No. 5,175,095, U.S. Pat. No. 5,420,034, U.S. Pat. No. 5,188,958, and U.S. Pat. No. 5,589,379.

When increased expression of the desaturase polypeptide in the source plant is desired, several methods can be employed. Additional genes encoding the desaturase polypeptide can be introduced into the host organism. Expression from the native desaturase locus also can be increased through homologous recombination, for example by inserting a stronger promoter into the host genome to cause increased expression, by removing destabilizing sequences from either the mRNA or the encoded protein by deleting that information from the host genome, or by adding stabilizing sequences to the mRNA (see U.S. Pat. No. 4,910,141 and U.S. Pat. No. 5,500,365.)

When it is desirable to express more than one different gene, appropriate regulatory regions and expression methods, introduced genes can be propagated in the host cell through use of replicating vectors or by integration into the host genome. Where two or more genes are expressed from separate replicating vectors, it is desirable that each vector has a different means of replication. Each introduced construct, whether integrated or not, should have a different means of selection and should lack homology to the other constructs to maintain stable expression and prevent reassortment of elements among constructs. Judicious choices of regulatory regions, selection means and method of propagation of the introduced construct can be experimentally determined so that all introduced genes are expressed at the necessary levels to provide for synthesis of the desired products.

Constructs comprising the gene of interest may be introduced into a host cell by standard techniques. These techniques include transfection, infection, biolistic impact, electroporation, microinjection, scraping, or any other method which introduces the gene of interest into the host cell (see U.S. Pat. No. 4,743,548, U.S. Pat. No. 4,795,855, U.S. Pat. No. 5,068,193, U.S. Pat. No. 5,188,958, U.S. Pat. No. 5,463,174, U.S. Pat. No. 5,565,346 and U.S. Pat. No. 5,565,347). For convenience, a host cell that has been manipulated by any method to take up a DNA sequence or construct will be referred to as “transformed” or “recombinant” herein. The subject host will at least have one copy of the expression construct and may have two or more, depending upon whether the gene is integrated into the genome, amplified, or is present on an extrachromosomal element having multiple copy numbers.

When necessary for the transformation method, the chimeric genes of the present invention can be inserted into a plant transformation vector, e.g. the binary vector described by Bevan (1984) Nucleic Acids Res., 12; 8711-8721. Plant transformation vectors can be derived by modifying the natural gene transfer system of Agrobacterium tumefaciens. The natural system comprises large Ti (tumor-inducing)-plasmids containing a large segment, known as T-DNA, which is transferred to transformed plants. Another segment of the Ti plasmid, the vir region, is responsible for T-DNA transfer. The T-DNA region is bordered by terminal repeats. In the modified binary vectors, the tumor inducing genes have been deleted and the functions of the vir region are utilized to transfer foreign DNA bordered by the T-DNA border sequences. The T-region also contains a selectable marker for antibiotic resistance, and a multiple cloning site for inserting sequences for transfer. Such engineered strains are known as “disarmed” A. tumefaciens strains, and allow the efficient transfer of sequences bordered by the T-region into the nuclear genome of plants.

Expression of a heterologous or reporter gene in developing seeds, young seedlings and mature plants can be monitored by immunological, histochemical, mRNA expression or activity assays. As discussed herein, the choice of an assay for expression of the chimeric gene depends upon the nature of the heterologous coding region. For example, Northern analysis can be used to assess transcription if appropriate nucleotide probes are available. If antibodies to the polypeptide encoded by the heterologous gene are available, Western analysis and immunohistochemical localization can be used to assess the production and localization of the polypeptide. Depending upon the heterologous gene, appropriate biochemical assays can be used. For example, acetyltransferases are detected by measuring acetylation of a standard substrate. The expression of a lipid desaturase gene can be assayed by analysis of fatty acid methyl esters (FAMES).

Another aspect of the present invention provides transgenic plants or progeny of these plants containing the chimeric genes of the invention. Both monocotyledonous and dicotyledonous plants are contemplated. Plant cells are transformed with the chimeric genes by any of the plant transformation methods described above. The transformed plant cell, usually in the form of a callus culture, leaf disk, explant or whole plant (via the vacuum infiltration method of Bechtold et al. (1993) C.R. Acad. Sci. Paris, 316; 1194-1199) is regenerated into a complete transgenic plant by methods well-known to one of ordinary skill in the art (e.g., Horsh et al., 1985). In a preferred embodiment, the transgenic plant is sunflower, soybean, maize, cotton, tobacco, peanut, flax, Arabidopisis, oil seed rape or other Brassica plant, or crambe. Since progeny of transformed plants inherit the chimeric genes, seeds or cuttings from transformed plants may be used to maintain the transgenic plant.

As sources of polypeptides having desaturase activity and oligonucleotides encoding such polypeptides are organisms which produce a desired poly-unsaturated fatty acid. As an example, microorganisms having an ability to produce ARA can be used as a source of Δ5-desaturase genes; microorganisms which GLA or SDA can be used as a source of Δ6-desaturase and/or Δ12-desaturase genes. Such microorganisms include, for example, those belonging to the genera Mortierella, Conidiobolus, Pythium, Phytophathora, Penicillium, Porphyridium, Coidosporium, Mucor, Fusarium, Aspergillus, Rhodotorula, and Entomophthora. Within the genus Porphyridium, of particular interest is Porphyridium cruentum. Within the genus Mortierella, of particular interest are Mortierella elongata, Mortierella exigua, Mortierella hygrophila, Mortierella ramanniana, var. angulispora, and Mortierella alpina. Within the genus Mucor, of particular interest are Mucor circinelloides and Mucor javanicus.

DNAs encoding desired desaturases can be identified in a variety of ways. As an example, a source of the desired desaturase, for example genomic or cDNA libraries from Mortierella, is screened with detectable enzymatically- or chemically-synthesized probes, which can be made from DNA, RNA, or non-naturally occurring nucleotides, or mixtures thereof. Probes may be enzymatically synthesized from DNAs of known desaturases for normal or reduced-stringency hybridization methods. Oligonucleotide probes also can be used to screen sources and can be based on sequences of known desaturases, including sequences conserved among known desaturases, or on peptide sequences obtained from the desired purified protein. Oligonucleotide probes based on amino acid sequences can be degenerate to encompass the degeneracy of the genetic code, or can be biased in favor of the preferred codons of the source organism. Oligonucleotides also can be used as primers for PCR from reverse transcribed mRNA from a known or suspected source; the PCR product can be the full length cDNA or can be used to generate a probe to obtain the desired full length cDNA. Alternatively, a desired protein can be entirely sequenced and total synthesis of a DNA encoding that polypeptide performed.

One aspect of the invention is the provision of a method by which a novel extract having valuable constituents can be obtained from the flax plant while avoiding thermal decomposition of the constituents to a major extent where possible. In some embodiments, this involves performing steps at a reduced temperature, such as below 20° C. In some embodiments, this involves using instruments or machines that do not produce increased temperatures, such as, for example, a silent bowl cutter, for the initial processing steps.

Extracting the proteins can be done with the aid of any means of extraction such as water, organic solvents or supercritical CO₂. One example of an organic solvent is ethanol. As already mentioned, the temperature during extraction and any subsequent stage required for removing the extractant at least in part, e.g. by distillation, should be 50° C. or lower and preferably 40° C. and lower to prevent thermal impairment of the constituents of the flowers. This means that in distillative separation the pressure must be reduced to the extent that the stated upper temperature limit can be maintained.

According to the invention the use of supercritical CO₂ for the extraction of proteins is particularly preferred since it can be carried out at low temperatures and thus particularly gently.

The extraction with supercritical CO₂ may be done in any apparatus suitable therefor. From the thermodynamic properties of CO₂, namely a critical temperature of 31.3° C. and a critical pressure of 71.5 bar, the lower limits for the temperature and for the pressure result for the extraction. In particular, working at a temperature of 40° C. or below is preferred for extraction with CO₂, the pressure being preferably in the range from 90 to 300 bar.

Extraction may be continued until all constituents extractable with supercritical CO₂ have been extracted from the plant or seeds. This is usually the case after a 1 to 2 hour duration of the extraction method. However, according to the invention it is also possible to extract only part of the constituents from the plant/seeds.

One advantage in using supercritical CO₂ as the extractant as compared to the use of other extractants, such as ethanol or water, is that extraction can be done at temperatures below 40° C., whilst e.g. conventional alcoholic extraction requires the ethanol to be distilled off at temperatures of more than 100° C.

In one embodiment, the proteins of the present invention are extracted using supercritical solvents, alone or mixed with a secondary solvent. The solvent used to carry out the extraction or extractions is selected from the group comprising water, alcohols, ketones, esters, ethers, polyhydric alcohols, chlorine solvents and mixtures of at least two of the aforementioned solvents.

Broadly speaking, the process involves contacting a protein, a protein solvent system and an antisolvent for the protein solvent system under conditions to at least partially dissolve the protein solvent system in the antisolvent with consequent precipitation of the protein. In one embodiment, the protein solvent system includes at least in part a halogenated organic alcohol, and preferably consists essentially of a single halogenated organic alcohol.

A wide variety of solvent/antisolvent precipitation processes can be used in accordance with the invention. For example, the GAS and PCA processes can be employed. Preferably however, the solvents of the invention are used in the PCA process wherein the protein is first dissolved in the solvent system, and then droplets of the solution are sprayed into an antisolvent under conditions to precipitate protein particles.

The preferred halogenated organic alcohols are the halogenated alkyl alcohols, especially the C₁-C₄ alcohols. Particular alcohol solvents are HFIP, trifluoroethanol, 2-chloroethanol and mixtures thereof. The single most preferred solvent is HFIP (CAS #920-66-1). This solvent has a boiling point of 59° C. and a density of 1.618 g/ml, and is very soluble in CO₂. Normally, only a single halogenated organic alcohol will be used as a protein solvent. However, multiple-component solvent systems can also be employed, so long as such systems include a halogenated organic alcohol as at least a part thereof.

A variety of antisolvents can also be used in the invention, such as CO₂, propane, butane, isobutane, nitrous oxide, sulfur hexafluoride, trifluoromethane, hydrogen and mixtures thereof. CO₂ is the most preferred antisolvent, owing to its low cost, ready availability and critical properties (Tc=81.0° C. and Pc=73.8 bar or 1070 psi). Furthermore, CO₂ is non-toxic, non-flammable, recyclable, and “generally regarded as safe” by the FDA and pharmaceutical industry.

During processing, the contact between the protein solution system and antisolvent is carried out at near or supercritical conditions for the antisolvent, e.g., from about 0.5-2 Pc and more preferably from about 0.9-1.5 Pc; when CO₂ is used as the antisolvent, pressure conditions are normally maintained at a level of from about 1000-14000 psig, and more preferably from about 1100-2800 psig. The temperature conditions during processing are generally relatively low in order to avoid heat denaturation of the protein. Generally, temperatures of up to about 60° C. and more preferably up to about 50° C. are used. When CO₂ is the antisolvent, such temperatures exceed the Tc.

In order to maximize production rates, the preferred process is carried out in a pressurized precipitation chamber equipped with a nozzle. The protein solution is sprayed through the nozzle into a precipitation zone containing the antisolvent. The resultant protein particles are collected in a downstream recovery filter, and can easily be further processed for pharmaceutical uses.

In most instances, the starting protein is dissolved in a halogenated organic alcohol solvent or solvent system containing such an alcohol, thereby producing true solutions. However, the invention is not so limited. That is, it is possible that the protein may be only partially dissolved or dispersed within the solvent. Therefore, as used herein, “solution” should be understood to mean not only true solutions but also partial solutions and dispersions. Similarly, while complete proteins are often processed in accordance with the invention, protein fragments or peptides could also be treated. Accordingly, the term “protein” refers to all types of proteinaceous species.

### EXAMPLES

These examples are for purposes of illustration and are not meant to limit the invention in any way.

### Examples 1-4

### Purification of Plant Protein Products

Plants are harvested
Whole plants or parts are placed in batches
A batch is placed into a silent bowl cutter with six vertically rotating scimitar knife blades and a horizontally rotating bowl. Between about 200# and about 600# of ice and between 4# and 20# of NaCl are added along with a biological buffer such as sodium phosphate that will mediate pH between about 6.5 and about 8.0. Comminuting is conducted for between about 20 and about 100 cycles, until particle size of between 1 and 0.5 millimeters is achieved. Protease inhibitors, including phenylmethylsulfonlfluoride (PMSF), p-aminobenzamidine, tosylamino-2-phenylethyl chloromethyl ketone (TPCK), and pepstatin A are added to Cf 0.001-1.0 mM.
The chopped mixture is then pumped into plastic tubes weighing between about 10# and about 30# and run through an immersion liquid N₂ freezer
Tubes are then stored in a holding freezer at between about −46° C. to about 0° C.
Tubes are removed and equilibrated to between about 5.5° C. and about 8.5° C.
Tubing is removed from crystallized liquid and material is placed into a critical CO₂ chamber operating between about 200 and about 2000 bars of pressure to effect complete cell breakage.
Material is processed between about 20 seconds and about 8 minutes
Material is removed from chamber and subjected to centrifugation at about 10K×g for about 10 min to remove unbroken material.
To the resulting solution, solid ammonium sulfate is slowly added to bring the solution to about 70% final concentration with stirring overnight at about 4° C., maintaining a constant pH of about 7.5 by addition of NaOH. The material is then held at about 4° C. for at least 3 days, allowing precipitated protein and debris to settle out of solution. Protein so precipitated is relatively insensitive to degradation and material at this stage (or after decantation, see below) may be held indefinitely at about 4° C.
Further processing: When convenient, the solution is carefully decanted or pumped out and discarded. Material remaining in the chamber is brought to a final concentration of about 33% ammonium sulfate with gentle agitation overnight in the presence of the same protease inhibitors as above.
A second settling process is allowed to occur over a period of 3 days and the solution (containing protein material (e.g., human serum albumin, HSA, produced in transgenic plants)) is carefully decanted and diluted to about 10% ammonium sulfate. This fluid is passed through a Sharples centrifuge to remove remaining particulate matter. The HSA is collected batch-wise on phenyl-acrylamide beads, followed by subsequent elution with phosphate-buffered saline.
Finally, the HSA is collected onto Cibacron blue F3GA-polystyrene beads batchwise and eluted with 1-4, preferably 3 sequential volumes of a solution containing about 0.25 M ammonium acetate, about pH 7.2 and lyophilized.

### Example 2

Plants are harvested
Whole plants or parts are placed are placed into a silent bowl cutter with six vertically rotating simitar knife blades and a horizontally rotating bowl. Between about 200# and about 600# of ice and between 4# and 20# of NaCl are added along with a biological buffer such as sodium phosphate that will mediate pH between about 6.5 and about 8.0. Comminutating is conducted for between about 20 and about 100 cycles, until particle size of between 1 and 0.5 millimeters is achieved. Protease inhibitors, including phenylmethylsulfonlfluoride (PMSF), p-aminobenzamidine, tosylamino-2-phenylethyl chloromethyl ketone (TPCK), and pepstatin A are added to Cf 0.001-1.0 mM.
The chopped mixture is then pumped into plastic tubes weighing between about 10# and about 30# and run through an immersion liquid N₂ freezer
Tubes are then stored in a holding freezer at between about −46° C. to about 0° C.
Tubes are removed and equilibrated to between about 5.5° C. and about 8.5° C.
Tubing is removed from crystallized liquid and material is placed into which supercritical CO₂ is introduced at a pressure between about 10 and about 2000 bars of pressure and between about 0° C. and about 80° C.
Material is processed between about 20 seconds and about 8 minutes
Material is removed from chamber and subjected to centrifugation at about 10K×g for about 10 min to remove unbroken material.
To the resulting solution, solid ammonium sulfate is slowly added to bring the solution to about 70% final concentration with stirring overnight at about 4° C., maintaining a constant pH of about 7.5 by addition of NaOH. The material is then held at about 4° C. for at least 3 days, allowing precipitated protein and debris to settle out of solution. Protein so precipitated is relatively insensitive to degradation and material at this stage (or after decantation, see below) may be held indefinitely at about 4° C.
Further processing: When convenient, the solution is carefully decanted or pumped out and discarded. Material remaining in the chamber is brought to a final concentration of about 33% ammonium sulfate with gentle agitation overnight in the presence of the same protease inhibitors as above.
A second settling process is allowed to occur over a period of 3 days and the solution (containing HSA) is carefully decanted and diluted to about 10% ammonium sulfate. This fluid is passed through a Sharples centrifuge to remove remaining particulate matter. The HSA is collected batch-wise on phenyl-acrylamide beads, followed by subsequent elution with phosphate-buffered saline.
Finally, the HSA is collected onto Cibacron blue F3GA-polystyrene beads batch-wise and eluted with 1-4, preferably 3 sequential volumes of a solution containing about 0.25 M ammonium acetate, about pH 7.2 and lyophilized.

### Example 3

Same as Example 2 above except that after placing in a chamber with supercritical CO₂, the treated material is passed into a separator and subjected to thermal debinding.

### Example 4

Same as Example 2 above except that after placing in a chamber with supercritical CO₂, the treated material is passed into a separator and subjected to non-thermal debinding.

In addition, information regarding procedural or other details supplementary to those set forth herein is described in cited references specifically incorporated herein by reference.

It will be apparent to those skilled in the art that modifications or variations may be made to the preferred embodiment described herein without departing from the novel teachings of the present invention. All such modifications and variations are intended to be incorporated herein and within the scope of the claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for recovering plant extract from plant material comprising:
chopping the plant material in a silent bowl cutter;
exposing the plant material to a solvent system;
freezing the chopped plant material in the solvent;
placing the chopped plant material in the solvent into a high pressure chamber;
increasing pressure in the chamber to about 50 to about 2000 bars;
removing mixture from the chamber; and
centrifuging mixture to separate out soluble material, including the plant extract in solvent.

2. The method of claim 1, wherein the method further comprises contacting the protein and solvent with an antisolvent for the protein solvent system under conditions to at least partially dissolve the protein solvent system in the antisolvent with consequent precipitation of the protein.

3. The method of claim 1, wherein the solvent system consists essentially of a single halogenated organic alcohol.

4. The method of claim 3, wherein the alcohol is a halogenated alkyl alcohol.

5. The method of claim 2, wherein the antisolvent is selected from the group consisting of CO₂, propane, butane, isobutane, nitrous oxide, sulfur hexafluoride, trifluoromethane, hydrogen, and mixtures thereof.

6. The method of claim 5, wherein the antisolvent is CO₂.

7. The method of claim 2, wherein the contact between the protein, solvent system and antisolvent is carried out at near or supercritical conditions for the antisolvent.

8. The method of claim 7, wherein the antisolvent is CO₂ and the total pressure is from about 90-300 bar.

9. The method of claim 1, wherein the chopping is performed at a temperature of less than about 20° C.

10. The method of claim 9, wherein the exposing the plant material to the solvent system is performed at a temperature of less than about 20° C.

11. The method of claim 1, wherein the plant extract in solvent comprises at least one protein.

12. The method of claim 11, wherein the at least one protein comprises human serum albumin.

13. The method of claim 12, wherein the protein in solvent is bound to a fatty acid.

14. The method of claim 1, wherein the solvent system is water.

15. The method of claim 14, wherein the solvent system is crushed ice.

16. The method of claim 1, wherein the chopping and exposing steps are performed at the same time.

17. The method of claim 1, further comprising adding ammonium sulfate to the plant extract in the solvent, following centrifugation.

18. The method of claim 17, wherein the chopping and exposing steps are performed at the same time.

19. The method of claim 13, wherein the fatty acid is DHA.
